(19) United States
(12) Patent Application Publication
(10) Pub. No.: US 2014/0031857 A1
(43) Pub. Date: Jan. 30, 2014

(54) EMBOLIC PROTECTION FILTER FOR TRANSCATHETER AORTIC VALVE REPLACEMENT AND USES THEREOF

(71) Applicant: BOSTON SCIENTIFIC SCIMED, INC., Maple Grove, MN (US)

(72) Inventor: Leonard B. Richardson, Brooklyn Park, MN (US)

(21) Appl. No.: 13/947,148

(22) Filed: Jul. 22, 2013

Related U.S. Application Data
(60) Provisional application No. 61/675,371, filed on Jul. 25, 2012.

(51) Int. Cl. A61F 2/01; A61F 2/24

(52) U.S. Cl. CPC A61F 2/013; A61F 2/24
USPC 606/200

(57) ABSTRACT

The disclosure pertains to an embolic protection device for use within the aorta which is well suited to prevent debris from entering the brachiocephalic artery, or right common carotid artery, the left common carotid, and the left subclavian artery during medical procedures within the heart while maintaining access to an interventional site within the heart and methods of use therefor. The embolic protection device also reduces release of debris into the downstream vasculature.

### RELATED APPLICATIONS

This applications claims the benefit of U.S. Provisional Application No. 61/675,371 filed Jul. 25, 2012.

### BACKGROUND

Preventing emboli and other debris from entering the carotid arteries (i.e., the brachiocephalic artery, or right common carotid artery, the left common carotid, and the left subclavian) by way of the aorta reduces the incidence of ischemic stroke. Emboli and other debris in the aorta come from several sources. These sources include: 1) aortic atheroma which detaches from the wall of the aorta due to various reasons including incising, clamping, and/or clamp release of the aorta during surgery; 2) debris released during surgery on the heart such as the installation of a replacement heart valve or to access the left atrial appendage; 3) thrombus which forms in the left atrium or the left atrial appendage resulting from atrial fibrillation; 4) thrombus which forms on ventricular assist devices; 5) venous thrombus which passes into the left ventricle through a patent foramen ovale or other arteriovenous shunt; and 6) other less common sources.

A variety of intravascular filtering means are known in the art and may consist of a flexible metallic grid, a flexible synthetic or plastic grid, a weave of synthetic filaments, or a nondegradable or possibly biodegradable textile cloth, often supported by a basket or funnel shaped frame which may be deployed within the lumen of a vessel to be protected.

There are fewer intravascular devices designed for arterial and especially aortic filtration. A filter that functions in arteries must address additional concerns because of the hemodynamic differences between arteries and veins. Arteries have thicker walls than veins to control higher average pressure and arterial blood flow is pulsatile with large pressure variations between systolic and diastolic flow. These pressure variations cause the artery walls to expand and contract. Thus, filters and diverters must be able to expand and contract along with the lumen of the aorta to which they may be anchored. Intravascular devices for aortic filtration and/or diversion of emboli typically occlude a significant portion of the lumen of the aorta rendering them unsatisfactory for use in combination with other intravascular devices which need to traverse the filter during valve replacement or other cardiac interventional procedures. In addition, the large volumetric flow through the aorta can rapidly clog most filters that attempt to filter 100% of the aortic blood flow.

The problem of preventing emboli from reaching the cerebral vasculature has thus far not been adequately addressed. Therefore, a need exists for new devices and methods to prevent embolic material from entering the carotid/cerebral arteries, while maintaining peripheral blood flow from the heart to the descending aorta.

### SUMMARY

This disclosure pertains to an embolic protection device comprising a conical filter element having a distal opening; an elongated filter wire disposed along a generatrix of the conical filter element; and a support structure fixedly attached to a distal end of the elongated filter wire and to the conical filter element at the distal opening thereof, wherein the support structure forms a partial circumferential arch along and attached to the distal opening, and further wherein the conical filter element includes a split distal region having an edge which together with the partial circumferential arch of the support structure defines a generally cylindrical passage through at least a portion of the conical filter element, said generally cylindrical passage lying parallel to a longitudinal axis of the conical filter element and along a wall of a vessel in which the embolic protection device is deployed.

The disclosure also pertains to a method of deploying a medical device comprising advancing a delivery catheter through the vasculature of a patient to a first deployment site; deploying an embolic protection device comprising a conical filter element having a distal opening, an elongated filter wire, and a support structure fixedly attached to a distal end of the elongated filter wire and to the conical filter element at the distal opening thereof at the first deployment site, wherein the conical filter element includes a split distal region an edge of which together with a partial circumferential arch of the support structure defines a generally cylindrical passage through at least a portion of the conical filter element; advancing an medical device through a patient's vasculature to a site proximal of the generally cylindrical passage through at least a portion of the conical filter element; advancing the medical device through the generally cylindrical passage; performing at least one of an interventional or a diagnostic procedure; removing the medical device from the generally cylindrical passage; recovering the embolic protection device; and removing the embolic protection device from the patient's vasculature.

### BRIEF DESCRIPTION OF DRAWINGS

FIG. 1 illustrates an embolic protection device of the disclosure.

FIG. 2 presents an axial view in the proximal direction of the embolic protection device of FIG. 1.

FIG. 3 illustrates the aortic arch and associated structures.

FIG. 4 illustrates an embolic protection device of the disclosure deployed in the aorta.

FIG. 5 illustrates an embolic protection device of the disclosure deployed in the aorta in conjunction with a second interventional device.

### DETAILED DESCRIPTION

The following description should be read with reference to the drawings wherein like reference numerals indicate like elements throughout the several views. The drawings, which are not necessarily to scale, are not intended to limit the scope of the claimed invention. The detailed description and drawings illustrate example embodiments of the claimed invention.

All numbers are herein assumed to be modified by the term “about.” The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect such feature, structure, or characteristic in connection with other embodiments, whether or not explicitly described, unless clearly stated to the contrary.

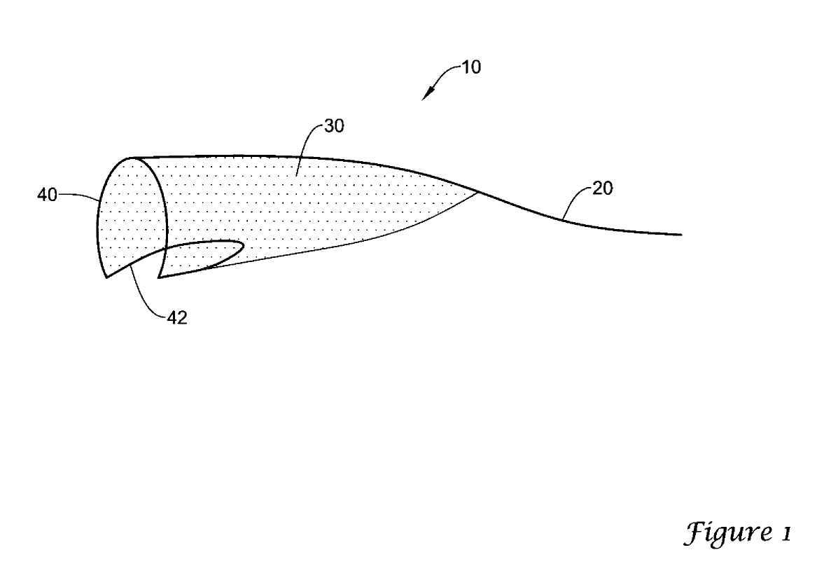

FIG. 1 illustrates an embodiment of embolic protection device 10 of the disclosure in which a conical filter element 30, comprising a membrane or mesh having a plurality of openings sized and adapted to permit blood cells to pass therethrough while preventing the passage of emboli and other debris, is attached at its open distal end to support structure 40 in the form of a partial circumferential arch. It will be appreciated that the conical filter element 30 need not be conical in the strict geometrical sense and may include, for example, a frustoconical region and/or one or more cylindrical regions of differing diameters. As used in the specification and claims, the terms “conical” and “generally conical” should be read broadly to include the variations described above and the like. Additionally, the conical filter element 30 may adopt a somewhat curved configuration to lie along the surface of the aortic arch. The conical filter element 30 may be attached to the support structure 40 by any of the methods employed in the art, such as adhesive bonding, thermal fusion, mechanical fasteners, sewing, and the like. The generally conical filter element 30 of the embolic protection device 10 is also fixedly attached to a distal region of an elongated filter wire 20 by similar methods. The filter wire is typically disposed along a generatrix of the generally conical filter element 30 and adapted to lie along the upper surface of the aorta. The use of an elongated filter wire 20 which is adapted to lie along the upper surface of the aorta provides good support and manipulability to the embolic protection device 10 without significant obstruction of the distal opening of the conical filter element or the interior thereof. The generally conical filter element 30 may be formed of materials employed for that purpose in the art such as, for example, a flexible metallic grid, a laser drilled membrane, a flexible synthetic or plastic grid, a weave of synthetic filaments, a nonwoven or a nondegradable or possibly biodegradable textile cloth.

In addition to being fixedly attached to the generally conical filter element 30, the elongated filter wire 20 is attached at its distal end to the support structure 40 by welding, soldering, winding, crimping, bonding, or the like. The elongated filter wire 20 may be formed from any of the solid or tubular materials or composites commonly employed for guidewires or filter wires and should have a length sufficient to reach from the deployment site, through the vasculature, and to extend beyond the introduction site on the patient's body, typically greater than the distance between the heart and the femoral artery.

The support structure 40 may be formed from an elastically recoverable material such as a biocompatible elastomeric polymer, spring steel, stainless steel, nickel-titanium alloy, or the like which can provide a self-opening function to the distal opening of the generally conical filter element 30 as well allowing the opening of the generally conical filter element 30 to flex as the vessel in which the embolic protection device 10 is deployed responds to pressure variations within the vessel. The self-expansion of the support structure 40 as well as the windsock-like inflation of the generally conical filter element 30 will typically ensure that the distal mouth opening of the embolic protection device will maintain contact with the surface of the aortic arch, thereby reducing the amount of debris which may bypass the generally conical filter element 30.

The generally conical structure of the filter element 30 is modified by the inclusion of a split distal extension of the distal opening of the filter element 30, said split distal extension of the opening defining an edge 42. Typically, the split distal extension will lie along the lower side of the filter element 30 in the deployed configuration of the filter element 30. In the discussion herein, reference numeral 42 may be used to refer to the edge of the opening and/or to a reinforcing member disposed along the edge depending on context. The addition of a split distal extension of the distal opening of the filter provides access for the passage of interventional or diagnostic medical devices and allows the distal opening of the filter element 30 to flex more freely thus accommodating the pulsate flow within the aorta. The edge 42 may include a reinforced portion of the generally conical filter element 30 which may be formed by folding and attaching the material of the conical filter element 30 at the edge 42 of the filter element 30, by coating the edge 42 with a stiffening material, by attaching a separate reinforcing member 42 in the form of a band to the region, and/or by providing a separate or integral extension of the support structure 40 which is attached to the edge in the region of the edge 42. In some embodiments, the support structure 40 and the reinforcing member 42 may be formed as a complete loop of a single material. In other embodiments, the reinforcing member may be attached along edge 42 and further attached at its ends to the ends of support structure 40. In yet other embodiments, the reinforcing member may be hingedly attached to the ends of support structure 40. In still other embodiments, the reinforcing member may include a flexible membrane or fringed portion (not shown) which extends into the split distal extension of the opening to provide an improved seal between the generally conical filter element 30 and the interventional device which passes through the split distal extension of the opening as described herein.

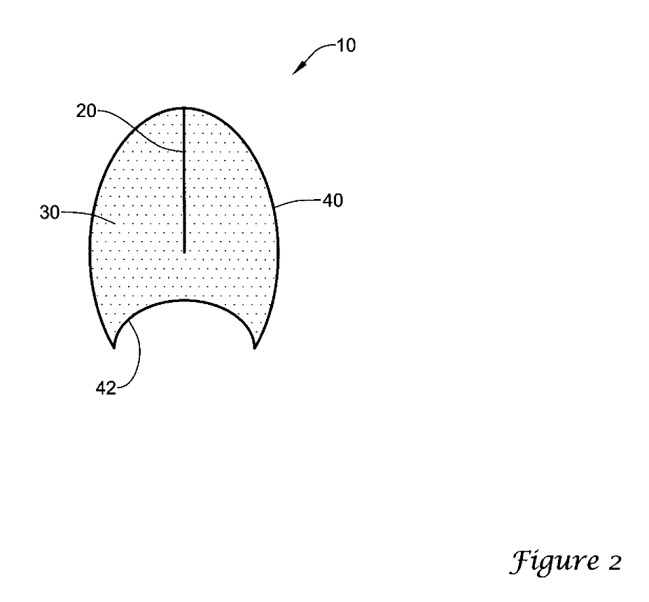

In each of these embodiments, the filter element 30, as modified by the inclusion of a split distal extension of the distal opening, will resemble a conical shell which has been intersected by a cylinder approximately parallel to the axis of the conical surface with edge 42 lying generally along the line of contact between the conical shell and the cylinder. When viewed axially through the distal opening of the filter element, the unconstrained embolic protection device 10 may appear as illustrated in FIG. 2; however when the embolic protection device 10 is constrained by a vessel in which the device is deployed, the edge 42, possibly in combination with a portion of the vessel wall, will typically appear more nearly circular. When the vessel in which the embolic protection device 10 is small, the ends of support structure 40 may touch, or even overlap, to form a nearly circular opening when viewed axially. It will be appreciated that the resulting circular or nearly circular opening may vary in diameter as the diameter of the vessel changes in response to pressure variations within the vessel.

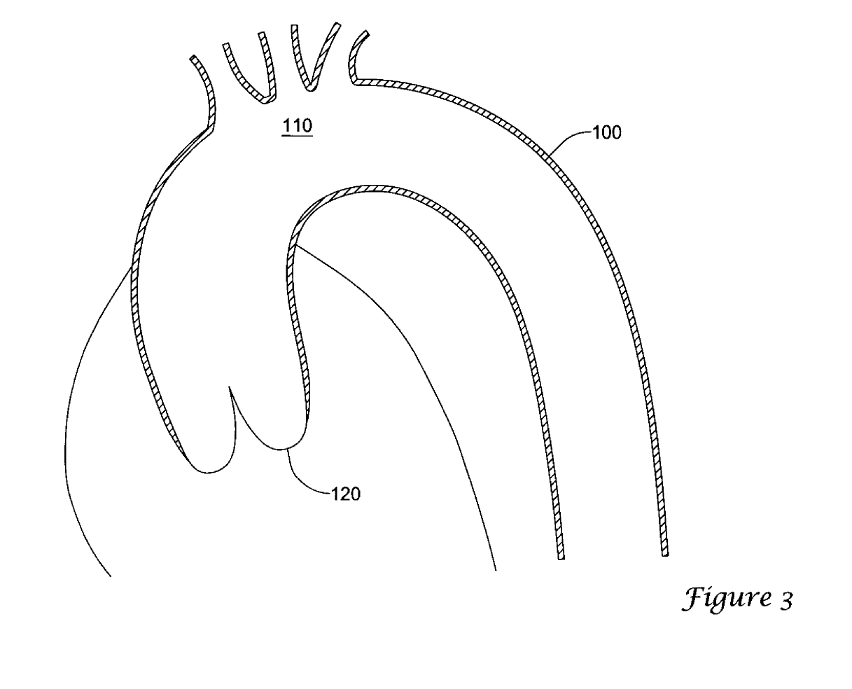

FIG. 3 illustrates an aorta 100 in which an embolic protection device of the disclosure may be deployed preparatory to performing, for example, a replacement of aortic valve 120. During such procedures, plaque and other debris may be generated which, if left unfiltered in region 110, may enter the brachiocephalic artery, or right common carotid artery, the left common carotid, and the left subclavian artery with attendant risk of ischemic stroke.

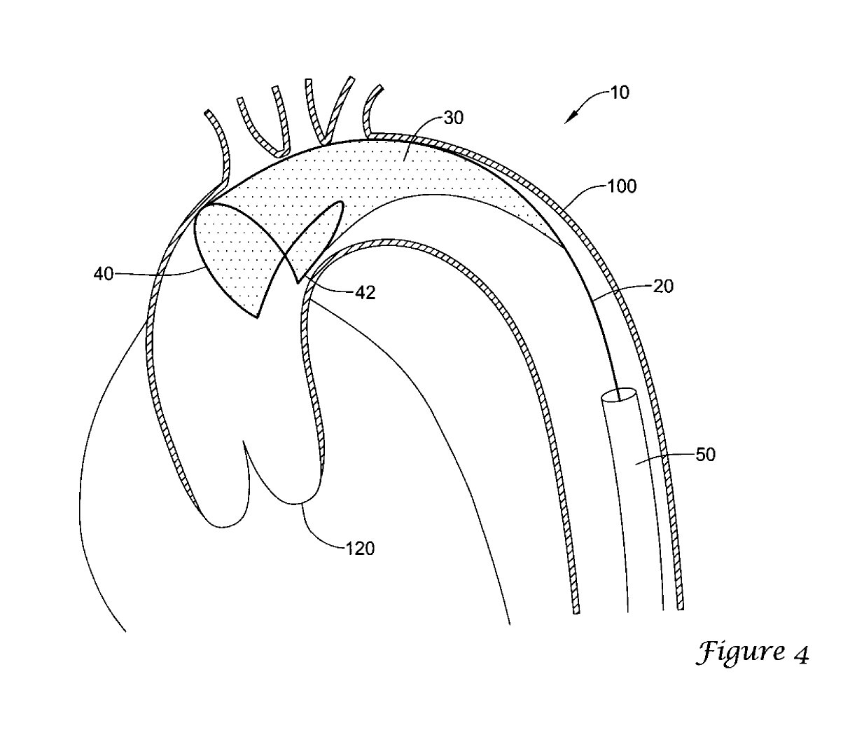

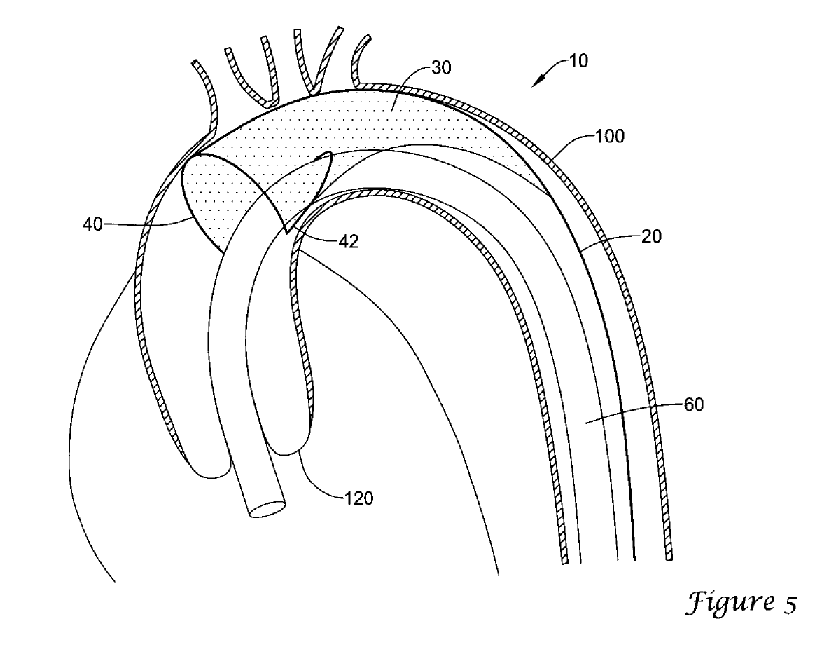

The deployment and use of an embolic protection device 10 of the disclosure is illustrated in FIGS. 4 and 5. In FIG. 4, the embolic protection device 10 has been advanced through the vasculature to the aorta 100 within a delivery catheter 50 and deployed therefrom by either advancing embolic protection device 10 relative to the delivery catheter 50; by withdrawing delivery catheter 50 relative to embolic protection device 10; or by a combination of these approaches such that the support structure 40 associated with the distal opening formed by conical filter element 30 is located between the aortic valve 120 and the brachiocephalic artery such that the conical filter element 30 substantially covers the region 110 of FIG. 3.

In some embodiments, a self-expanding support structure 40 may recover elastically upon deployment of the embolic protection device 10 from the delivery catheter to press the distal opening formed by conical filter element 30 against the wall of the aorta 100 while the gap in the support structure 40 allows the distal opening to expand and contract in the region of contact to maintain a seal between the conical filter element 30 and the surface of the aorta 100. In other embodiments, the support structure 40 may play a more passive role in which it tends to maintain the perimeter of the distal opening of the conical filter element 30 in an extended and fold-free state while inflation of the windsock-like conical filter element 30 by blood flow within the aorta 100 suffices to ensure that the distal region of contact maintains a seal between the conical filter element 30 and the wall of the aorta 100. Once the embolic protection device 10 is properly positioned, delivery catheter 50 may be removed.

It will be appreciated that radiopaque and/or MRI markers (not shown) associated with the embolic protection device 10 and particularly with the support structure 40 and or a distal region of elongate filter wire 20 may be useful in determining if the deployed embolic protection device 10 is properly located within the aorta 100. Additionally, the elongate filter wire 20 and/or the conical filter element 30 may be provided with radial protrusions which may engage any of the brachiocephalic artery, or right common carotid artery, the left common carotid, and the left subclavian artery to further locate and stabilize the position of the embolic protection device 10 within the aorta 100. If it is determined that the location of the deployed embolic protection device 10 is less that optimal, delivery catheter 50 may be advanced relative to the elongate filter wire 20 and conical filter element 30 to recapture the embolic protection device 10, whereupon the embolic protection device 10 may be repositioned and redeployed.

Once the delivery catheter 50 has been removed, a significant fraction of the cross- sectional area of the aorta 100 remains unobstructed while the embolic protection device 10 effectively both diverts debris from the brachiocephalic artery, or right common carotid artery, the left common carotid, and the left subclavian artery and captures debris within the conical filter element 30 thereby protecting downstream tissue from damage.

In FIG. 5, a medical device 60 such as a delivery system for a replacement heart valve has been advanced through the aorta and generally along the cylindrical path created by the edge 42 of split distal extension of the conical filter element 30 and possibly a portion of the support structure 40 and/or the wall of the aorta 100. A reinforcing member associated with edge 42 may help to define the cylindrical path and/or provide a supplemental seal against the medical device 60. In addition to a delivery system for a replacement heart valve, the medical device may be any of a valvuloplasty catheter, a left atrial appendage plug delivery system, or other interventional or diagnostic device 60 which commonly might be advanced through the aortic valve 120.

Upon completion of the procedure, medical device 60, less any portion thereof which has been implanted, may be withdrawn along the cylindrical path without dislodging emboli or other debris trapped near the apex of the conical filter element 30. Following the removal of medical device 60, delivery catheter 50 or an equivalent retrieval device may be (re)inserted and advanced to collapse the conical filter element 30 and subsequently may be removed with the filter element 30 and captured debris from the patient's body in a reversal of the illustrated deployment process.

Although the illustrative examples described above relate to placement within the aorta for protection during heart surgery, placement in other locations is also contemplated, particularly when the preferred direction of insertion of the interventional or diagnostic device is from a point downstream of the site of the intervention and when it is desirable to avoid occlusion of the vessel lumen by filter support structures. In such an embodiment, the dimensions of the conical filter element and the lengths of the filter wire and delivery catheter may be adjusted to better suit the local anatomy of the deployment site.

Various modifications and alterations of this invention will become apparent to those skilled in the art without departing from the scope and principles of this invention, and it should be understood that this invention is not to be unduly limited to the illustrative embodiments set forth hereinabove. All publications and patents are herein incorporated by reference to the same extent as if each individual publication or patent was specifically and individually indicated to be incorporated by reference.

What is claimed is:

1. An embolic protection device comprising:
a conical filter element having a distal opening;
an elongated filter wire disposed along a generatrix of the conical filter element; and
a support structure fixedly attached to a distal end of the elongated filter wire and to the conical filter element at the distal opening thereof,
wherein the support structure forms a partial circumferential arch along and attached to the distal opening, and
further wherein the conical filter element includes a split distal region having an edge which together with the partial circumferential arch of the support structure defines a generally cylindrical passage through at least a portion of the conical filter element, said generally cylindrical passage lying parallel to a longitudinal axis of the conical filter element and along a wall of a vessel in which the embolic protection device is deployed.

2. The embolic protection device of claim 1, wherein the split distal region of the conical filter element includes a reinforcing member along the edge thereof

3. The embolic protection device of claim 2, wherein the reinforcing member is a split support attached at ends thereof to the ends of the partial circumferential arch of the support structure.

4. The embolic protection device of claim 2, wherein the reinforcing member and the partial circumferential arch of the support structure form an integral wire loop.

5. The embolic protection device of claim 1, wherein the partial circumferential arch of the support structure is adapted to lie along the wall of the vessel in which the embolic protection device is deployed and to flex while maintaining contact with the vessel wall as radial dimensions of the vessel wall change in response to pressure variations within the vessel.

6. The embolic protection device of claim 1, wherein the partial circumferential arch of the support structure is formed of an elastically recoverable material.

7. The embolic protection device of claim 6, wherein the elastically recoverable material is a superelastic material.

8. The embolic protection device of claim 7, wherein the superelastic material is a nickel-titanium alloy.

9. The embolic protection device of claim 1, wherein the conical filter element comprises a membrane having a plurality of holes therethrough sized and adapted to allow passage of blood cells while retaining emboli and other debris which are larger than blood cells.

10. The embolic protection device of claim 1, wherein the conical filter element comprises a mesh having a plurality of holes therethrough sized and adapted to allow passage of blood cells while retaining emboli and other debris which are larger than blood cells.

11. The embolic protection device of claim 1, further comprising a delivery catheter.

12. The embolic protection device of claim 1, wherein the generally cylindrical passage through at least a portion of the conical filter element is sized and adapted to accommodate a medical device.

13. The embolic protection device of claim 12, wherein the medical device is an interventional device.

14. The embolic protection device of claim 12, wherein the medical device is a diagnostic device.

15. A method of deploying a medical device comprising:
advancing a delivery catheter through the vasculature of a patient to a first deployment site;
deploying an embolic protection device comprising a conical filter element having a distal opening, an elongated filter wire, and a support structure fixedly attached to a distal end of the elongated filter wire and to the conical filter element at the distal opening thereof at the first deployment site,
wherein the conical filter element includes a split distal region an edge of which together with a partial circumferential arch of the support structure defines a generally cylindrical passage through at least a portion of the conical filter element;
advancing a medical device through a patient's vasculature to a site proximal of the generally cylindrical passage through at least a portion of the conical filter element;
advancing the medical device through the generally cylindrical passage; performing at least one of an interventional or a diagnostic procedure;
removing the medical device from the generally cylindrical passage;
recovering the embolic protection device; and
removing the embolic protection device from the patient's vasculature.

16. The method of deploying a medical device of claim 15, wherein the embolic protection device further includes a reinforcing member along the edge of the split distal region.

17. The method of deploying a medical device of claim 15, wherein generally cylindrical passage is sized and adapted to substantially conform to the medical device.

18. The method of deploying a medical device of claim 15, wherein the embolic protection device is sized and adapted to be deployed in an aorta.

19. The method of deploying a medical device of claim 18, wherein deploying the embolic protection device locates the support structure between an aortic valve and an ostium of a brachiocephalic artery.

20. The method of deploying a medical device of claim 15, wherein the medical device is a replacement heart valve.
